(19) United States
(12) Patent Application Publication
(10) Pub. No.: US 2021/0278382 A1
(43) Pub. Date: Sep. 9, 2021

(54) MEASUREMENT APPARATUS FOR GAS SENSOR

(71) Applicant: EPISTAR CORPORATION, Hsinchu (TW)

(72) Inventor: Wei-Chih PENG, Hsinchu (TW)

(21) Appl. No.: 16/836,781

(22) Filed: Mar. 31, 2020

(30) Foreign Application Priority Data
Mar. 5, 2020 (TW) 109202472

(51) Int. Cl. G01N 33/00; G01R 31/28; G01R 1/04

(52) U.S. Cl. CPC G01N 33/007; G01R 1/0408; G01R 31/2831

(57) ABSTRACT

A measurement apparatus for gas sensor includes a wafer-holding module and a vacuuming module. The wafer-holding module includes a holding carrier configured to hold a wafer. The holding carrier includes an uppermost surface, a bottommost surface, an outermost side surface between the uppermost surface and the bottommost surface, a plurality of grooves, and a plurality of through holes. The vacuuming module couples to the plurality of through holes and is configured to generate a negative pressure to attach the gas-sensing cell to the holding carrier. The wafer includes at least one uncut gas-sensing cell. At least one gas-sensing cell has a cavity located right above at least one of the grooves. The grooves extend downwardly from the uppermost surface and not reaching the bottommost surface. The grooves extend outwardly in a horizontal direction to expose out of the outermost side surface.

### CROSS-REFERENCE TO RELATED APPLICATION

This application claims priority to and the benefit of Taiwan Patent Application No. 109202472 filed on Mar. 5, 2020, which is incorporated by reference herein in its entirety.

### BACKGROUND

### Technical Field

This disclosure relates to a measurement apparatus for gas sensor, in particular, to a measurement apparatus of measuring a gas-sensing cell in its wafer form.

### Related Art

To test the function of a gas sensor, the gas sensor has to be operated under an environment having a specific gas with a certain concentration. In general, the testing is performed after the gas sensor is packaged.

However, as realized by the inventor, generally speaking, in the manufacturing process of a semiconductor type gas sensor-(e.g., a micro-electromechanical system (MEMS) type), a piece of wafer may include more than thousands or ten thousands of uncut gas-sensing cells. The uncut gas-sensing cells have to be separated from each other and packaged. The foregoing tests are then applied to each of the packaged gas-sensing cells. As a result, the testing procedure is time-consuming. Moreover, as the defects of the gas-sensing cells cannot be repaired by subsequent cutting and packaging procedures, the subsequent cutting and packaging procedures applied to the defected cells are time consuming and increases the production costs.

In view of this, according to one or some embodiments of the instant disclosure, a measurement apparatus for gas sensor is provided, and the measurement apparatus is adapted to measure gas-sensing cells in their wafer form.

### SUMMARY

In one embodiment, a measurement apparatus for gas sensor includes a wafer-holding module and a vacuuming module. The wafer-holding module includes a holding carrier configured to hold a wafer. The holding carrier includes an uppermost surface, a bottommost surface, an outermost side surface between the uppermost surface and the bottommost surface, a plurality of grooves, and a plurality of through holes. The wafer includes at least one uncut gas-sensing cell. At least one gas-sensing cell has a cavity located right above at least one of the grooves. The grooves extend downwardly from the uppermost surface and not reaching the bottommost surface. The grooves extend outwardly in a horizontal direction to expose out of the outermost side surface. The vacuuming module couples to the plurality of through holes, and is configured to attach the gas-sensing cell to the holding carrier.

Detailed description of the characteristics and the advantages of the instant disclosure are shown in the following embodiments. The technical content and the implementation of the instant disclosure should be readily apparent to any person skilled in the art from the detailed description, and the purposes and the advantages of the instant disclosure should be readily understood by any person skilled in the art with reference to content, claims, and drawings in the instant disclosure.

### BRIEF DESCRIPTION OF THE DRAWINGS

The disclosure will become more fully understood from the detailed description given herein below for illustration only, and thus not limitative of the disclosure, wherein:

FIG. 1 illustrates a side view of a measurement apparatus for gas sensor according to an exemplary embodiment of the instant disclosure;

FIG. 2 illustrates a perspective view of a measurement apparatus for gas sensor according the exemplary embodiment;

FIG. 2A illustrates an enlarged partial top perspective view of a holding carrier according to the embodiment shown in FIG. 1;

FIG. 2B illustrates an enlarged partial bottom perspective view of a wafer according to the embodiment shown in FIG. 1;

FIG. 3 illustrates a side view of a measurement apparatus for gas sensor according to another exemplary embodiment of the instant disclosure;

FIG. 4 illustrates a perspective view of the measurement apparatus for gas sensor according to the embodiment shown in FIG. 3; and

FIG. 5 illustrates an enlarged partial perspective view of a probe module of the measurement apparatus for gas sensor according to the embodiment shown in FIG. 3.

### DETAILED DESCRIPTION

Embodiments are provided, along with the figures, for facilitating the descriptions of the instant disclosure. It is understood that, a plenty of details are provided for readers to understand the disclosure; however, the inventions of the disclosure are still implementable in the premise that some or all of the details are omitted. In all the figures, same reference numbers designate identical or similar elements. It is worthy to mention that, the figures are provided for illustrative purposes, and are not used to indicate the actual size or number of the element. Moreover, some details may be omitted in the drawings for the sake of clarity for the drawings.

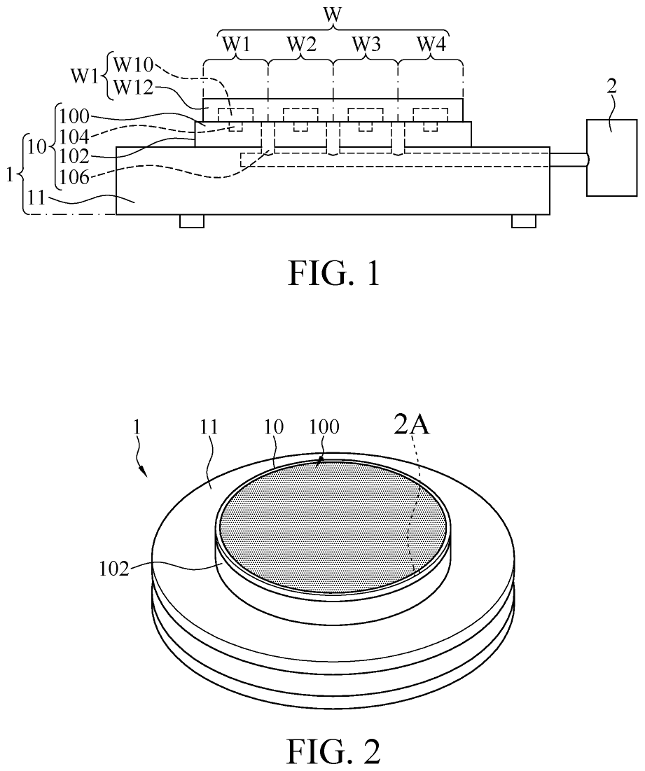

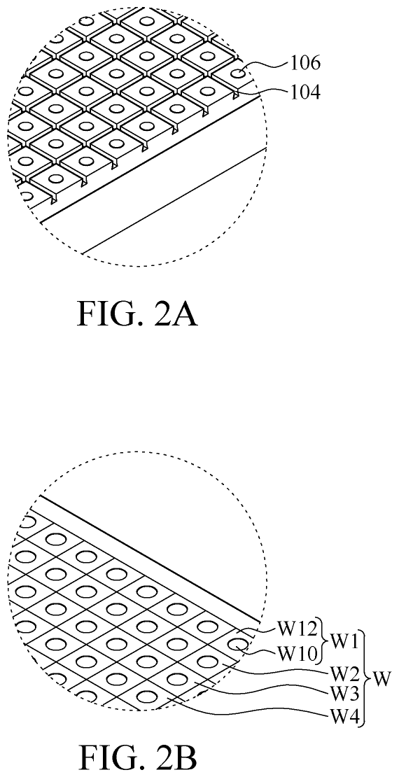

FIG. 1 illustrates a side view of a measurement apparatus for gas sensor according to an exemplary embodiment of the instant disclosure. FIG. 2 illustrates a perspective view of a measurement apparatus for gas sensor according to the exemplary embodiment. FIG. 2A illustrates an enlarged partial perspective view of a holding carrier according to the embodiment shown in FIG. 1, in a top perspective view. FIG. 2B illustrates an enlarged partial bottom perspective view of a wafer according to the embodiment shown in FIG. 1.

Please refer to FIGS. 1 to 2B, a measurement apparatus for gas sensor according to an exemplary embodiment of the instant disclosure is illustrated, and the measurement apparatus is adapted to measure a wafer-type gas-sensing cell (that is, the cutting process is not performed on wafer). For example, the measurement apparatus is adapted to measure a wafer W having several gas-sensing cells W1, W2, W3, W4. In this embodiment, the measurement apparatus for gas sensor comprises a wafer-holding module 1 and a vacuuming module 2. The wafer-holding module 1 comprises a holding carrier 10. A substrate 11 is located below the holding carrier 10, and the holding carrier 10 is adapted to hold the wafer W. The wafer W comprises at least one uncut gas-sensing cell W1, W2, W3, W4. For example, the wafer-holding module 1 may be, but not limited to, a chuck.

The holding carrier 10 has an uppermost surface 100, a bottommost surface, an outermost side surface 102, at least one groove 104, and at least one through hole 106. The outermost side surface 102 connects the uppermost surface 100 and the bottommost surface. In this embodiment, the profile of the holding carrier 10 is a circle, the uppermost surface 100 is a circular plane, and the outermost side surface 102 surrounds the uppermost surface 100 to form an annular side surface, but embodiments are not limited thereto.

The grooves 104 are located on the uppermost surface 100 and recessed downwardly, and the grooves 104 are extending outwardly and horizontally to the outermost side surface 102 in communication with outside environments. In other words, the grooves 104 are open channels, rather than hermetic ones. The grooves 104 are extending downwardly from the uppermost surface 100 and not reaching the bottommost surface. Moreover, the grooves 104 are arranged to pass through the bottom portions of the gas-sensing cells W1, W2, W3, W4, so that the cavities W10 inside the gas-sensing cells W1, W2, W3, W4 are respectively in communication with the grooves 104. Hence, the cavities W10 inside the gas-sensing cells W1, W2, W3, W4 are in communication with the outside environments. The gas pressure among the cavities W10, the grooves 104, and the outside environment can be balanced. In one embodiment, the grooves 104 are stripe trenches, but embodiments are not limited thereto.

The through holes 106 are located on the uppermost surface 100 of the holding carrier 10 and through the holding carrier 10 but not in communication with outside environment. In one embodiment, the shape of the cross section area of the through hole 106 is circular or rectangular, but embodiments are not limited thereto. The vacuuming module 2 may be in direct or indirect communication with the through holes 106. A negative pressure is generated in the through hole 106 through the vacuuming module 2, so that annular walls W12 of the gas-sensing cells W1, W2, W3, W4 are sucked on the holding carrier 10. In one embodiment, the vacuuming module 2 has a pump and a vacuum pipe assembly, but embodiments are not limited thereto.

Accordingly, when the gas-sensing cells W1, W2, W3, W4 of the wafer W are placed on the holding carrier 10, negative pressures are formed in the through holes 106 due to the suction caused by the vacuuming module 2, and the annular walls W12 of the gas-sensing cells W1, W2, W3, W4 are sucked on the holding carrier 10. Hence, the wafer W can be held on the holding carrier 10. Moreover, the gas pressures within the cavities W10 of the gas-sensing cells W1, W2, W3, W4 and outside the environment can be adjusted to be balanced or close to each other through the grooves 104 in communication the cavity and the outside environment. As a result, the cavities W10 can be immune from becoming hermetic spaces which have different gas pressures from the exterior pressure. The pressure difference can further bring damage(s) and failure(s) to the gas-sensing cells W1, W2, W3, W4. Moreover, a more accurate measurement result can be obtained by testing the gas-sensing cells W1, W2, W3, W4 under a condition of balanced or quasi-balanced pressure.

Furthermore, according to one or some embodiments of the instant disclosure, the measurement apparatus for gas sensor is adapted to perform a wafer-scale measurement to the gas-sensing cells which are not cut and not packaged. Hence, the characteristic parameters and the defect-free rate of the gas-sensing cells may be measured at the wafer stage, and the defective cells can be filtered and do not need to be performed subsequent procedures. Therefore, the overall manufacturing costs can be effectively reduced and the product reliability can be ensured.

In at least one embodiment, a surface area of the holding carrier 10 is greater than that of an object to be measured; for example, the surface diameter of the holding carrier 10 may be, but not limited to, greater than 300 mm. In one embodiment, the arrangement of the through holes 106 and the grooves 104 are staggered on the holding carrier 10. For example, the grooves 104 of the holding carrier 10 may be arranged in a two-dimensional array, and the through holes 106 are arranged at center portions of plural rectangular regions enclosed by the two-dimensional array.

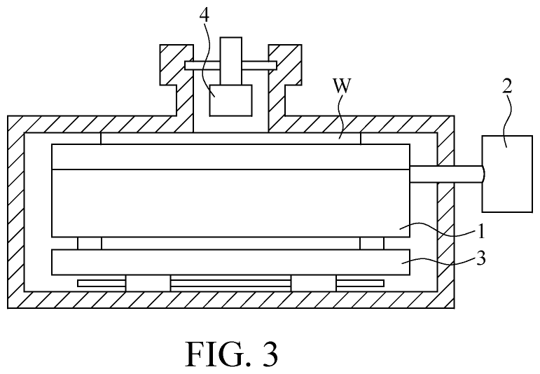

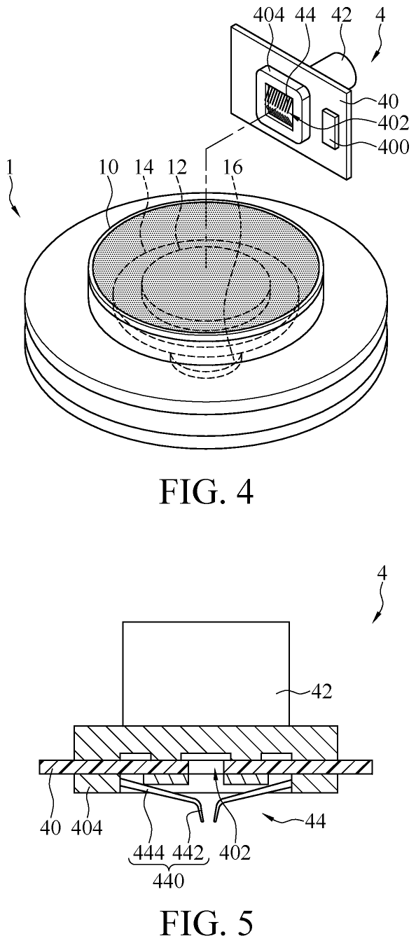

FIG. 3 illustrates a side view of a measurement apparatus for gas sensor according to another exemplary embodiment of the instant disclosure. FIG. 4 illustrates a perspective view of the measurement apparatus for gas sensor of the embodiment shown in FIG. 3. FIG. 5 illustrates an enlarged partial perspective view of a probe module of the measurement apparatus for gas sensor of the embodiment shown in FIG. 3.

In one embodiment, as shown in FIG. 3, the measurement apparatus for gas sensor further includes a driving platform 3 (in this embodiment, an XYZ stage). The driving platform 3 is connected to the wafer-holding module 1, and the driving platform 3 is adapted to drive the wafer-holding module 1 to move along the three-dimensional directions, so that the positions of the gas-sensing cells W1, W2, W3, W4 within the wafer W can be changed. For example, the driving platform 3 may comprise a stepwise motor and a transmission assembly, but embodiments are not limited thereto.

In another embodiment, with reference to FIGS. 3 and 4, the measurement apparatus for gas sensor further includes a heating unit 12, a temperature-sensing unit 14, and a temperature controlling unit 16. The heating unit 12 is placed adjacent to the holding carrier 10, and the heating unit 12 is adapted to heat the holding carrier 10 according to a temperature control signal. For example, the heating unit 12 may be a heat pipe which is made of a metal material having an impedance not prone to be interfered by testing gas, but embodiment are not limited thereto. The holding carrier 10 receives the heat through the heating unit 12 and transmits the heat to the sensing material on the gas-sensing cells W1, W2, W3, W4. The gas-sensing cells W1, W2, W3, W4 can therefore be heated to an required operating temperature range, e.g., 180 to 250 Celsius degrees, to make the sensing material to react with the testing gas and facilitate the electrical property measurement and function verification.

The temperature sensing unit 14 is coupled to the holding carrier 10. The temperature sensing unit 14 is adapted to sense the temperature of the holding carrier 10 to obtain a surface temperature of the uppermost surface 100 and output a corresponding temperature parameter/data. The temperature controlling unit 16 is electrically connected to the heating unit 12 and the temperature sensing unit 14. The temperature controlling unit 16 receives the temperature data measured by the temperature sensing unit 14 and compares the temperature data with a predetermined temperature data. When the surface temperature of the uppermost surface 100 is lower than the predetermined temperature data which is suitable for the operation of the gas-sensing cells W1, W2, W3, W4, the temperature controlling unit 16 outputs a temperature rising signal to the heating unit 12, so that the heating unit 12 provides more heats to the holding carrier 10. Conversely, when the surface temperature of the uppermost surface 100 is greater than the predetermined temperature data which is suitable for the operation of the gas-sensing cells W1, W2, W3, W4, the temperature controlling unit 16 outputs a temperature deceasing signal to the heating unit 12, so that the operation of the heating unit 12 is paused.

Based on the foregoing embodiment, the measurement apparatus for gas sensor has the wafer-holding module 1 integrated with a thermal control device. Hence, the thermal control device heats the wafer W to the operation temperature of the gas-sensing cells W1, W2, W3, W4 for measuring the gas-sensing cells W1, W2, W3, W4, and adjusting the heating temperature by monitoring the temperature of wafer W.

In one embodiment, with reference to FIGS. 3 to 5, the measurement apparatus for gas sensor further includes a probe module 4. The probe module 4 is arranged to face the wafer-holding module 1. The probe module 4 includes a circuit board 40, a gas inlet channel 42, and a pin structure 44.

The circuit board 40 has a control circuit 400, a hole 402, and a heat conduction ring 404. The circuit board 400 is electrically connected to the pin structure 44, and the control circuit 400 is adapted to process the electrical signals measured by the pin structure 44. From a top perspective view, the heat conduction ring 404 encloses the hole 402 and is connected to the circuit board 40. The heat conduction ring 404 is adapted to take the heats on the circuit board 40 away from the probe module 4.

The heat conduction ring 404 of the circuit board 40 is electrically insulated and has a good thermal conductivity. For example, the heat conduction ring 404 may be, but not limited to, made of an epoxy resin. In an example, it is understood that the operation temperature of the cells to be measured (e.g., the gas-sensing cells W1, W2, W3, W4) is near to 200 Celsius degrees, and the circuit board 40 of the probe module 4 is a printed circuit board (PCB) which can sustain a temperature not greater than about 150 Celsius degrees. The heats come from the pin structure 44 can be transmitted away from the circuit board 40 by the heat conduction ring 404, so that the circuit board 40 can be protected from being damaged by the high temperature and the probe module 4 can be kept to be operated normally.

In another embodiment, the measurement apparatus for gas sensor can be applied in measuring gases, such as hydrogen (H₂), hydrogen sulfide (H₂S), ammonia (NH₃), ethanol (C₂H₅OH), and carbon monoxide (CO). The measurement apparatus may be used along with a flow controller to adjust the concentration of the testing gas. The gas inlet channel 42 is in communication with the hole 402, and the gas inlet channel 42 is adapted to allow the testing gas to flow into the measurement apparatus. The testing gas flows toward the holding carrier 10 through the hole 402. After the testing gas reacts with the sensing material on the surfaces of the gas-sensing cells W1, W2, W3, W4, the gas-sensing cells W1, W2, W3, W4 can react with the testing gas and output the characteristic parameters of the testing gas. Accordingly, the characteristic parameters of the measured gas-sensing cells W1, W2, W3, W4 are compared with the reference parameters of a standard sample to determine the quality of the gas-sensing cells W1, W2, W3, W4.

As shown in FIG. 5, the pin structure 44 comprises a plurality of probes 440, the probes 440 are spaced from each other and disposed oppositely. The probe module 4 probes the uncut gas-sensing cells W1, W2, W3, W4 belonged to the wafer W with the plurality of probes 440 at the same time. Therefore, several cells may be probed at one time. The probes 440 are exposed out of the hole 402 and of cantilever configurations. The probes 440 in cantilever configurations can achieve a proper probing contact even if the surface of the wafer W is uneven. The measurement difficulty coming from the wafer warpage is therefore overcome. The probe 440 has a tip portion 442 and a cantilever portion 444. The tip portion 442 is adapted to detect a surface electrode of the gas-sensing cells W1, W2, W3, W4. The tip portion 442 can be used to heat conduction to reduce the temperature of the gas-sensing cells W1, W2, W3, W4. The cantilever portion 444 directly contacts the heat conduction ring 404, and the cantilever portion 444 transmits heats from the tip portion 442 away from the circuit board 40 through the heat conduction ring 404.

Based on the foregoing embodiment, the measurement apparatus for wafer-scale gas sensor can detect several cells at one time. Therefore, a high quality electrical property measurement and quality sorting can be provided, and the measurement efficiency can be improved.

In one embodiment, the probes 440 may be made of a tungsten material, which is suitable for testing the corrosive gas or the wafer W with a high operation temperature. In another embodiment, each of the probes 440 further includes a protection layer, and the protection layer is made of a fluoro material for isolating the corrosive gas.

Based on the above, according to one or some embodiments of the instant disclosure, a measurement apparatus for gas sensor is suitable for measuring the wafer-type gas sensor cells by using the through holes 106 to generate a vacuum suction force to hold the wafer-type gas-sensing cells. The grooves 104 are used to balance the internal gas pressures of the gas-sensing cells and the outside environmental. Hence, the measurement apparatus can get the accurate measurement results. Accordingly, the performance of the gas-sensing cells can be measured at the wafer stage in advance, and the data can be feedback to the research and development engineers. Therefore, the time duration and the cost for research and development can be reduced. Moreover, regarding the production, since the electrical property of the cells can be detected at the wafer stage in advance, the defect cells can be prevented from processing subsequent procedures. Therefore, the overall manufacturing costs can be effectively reduced, making the product more competitive.

While the instant disclosure has been described by the way of example and in terms of the preferred embodiments, it is to be understood that the invention need not be limited to the disclosed embodiments. On the contrary, it is intended to cover various modifications and similar arrangements included within the spirit and scope of the appended claims, the scope of which should be accorded the broadest interpretation so as to encompass all such modifications and similar structures.

What is claimed is:

1. A measurement apparatus for gas sensor, comprising:
a wafer-holding module, comprising:
a holding carrier configured to hold a wafer and comprising an uppermost surface, a bottommost surface, an outermost side surface between the uppermost surface and the bottommost surface, a plurality of grooves, and a plurality of through holes, wherein the wafer comprises at least one uncut gas-sensing cell; and
a vacuuming module coupling to the plurality of through holes, and configured to generate a negative pressure to attach the at least one uncut gas-sensing cell to the holding carrier,
wherein the at least one uncut gas-sensing cell has a cavity located right above at least one of the grooves, and
wherein the grooves extend downwardly from the uppermost surface and not reaching the bottommost surface, and the grooves extend outwardly in a horizontal direction to expose out of the outermost side surface.

2. The measurement apparatus for gas sensor according to claim 1, further comprising a driving platform connected to the wafer-holding module, wherein the driving platform is adapted to move the wafer-holding module.

3. The measurement apparatus for gas sensor according to claim 1, wherein the at least one uncut gas-sensing cell is distributed over the wafer.

4. The measurement apparatus for gas sensor according to claim 1, wherein the plurality of through holes and the plurality of grooves are alternately arranged on the holding carrier.

5. The measurement apparatus for gas sensor according to claim 1, wherein the plurality of grooves are arranged in a two dimensional array.

6. The measurement apparatus for gas sensor according to claim 1, further comprising:
a heating unit adjacent to the holding carrier, wherein the heating unit is adapted to heat the holding carrier according to a temperature control signal;
a temperature sensing unit coupled to the holding carrier, and used to output temperature data; and
a temperature controlling unit electrically connected to the heating unit and the temperature sensing unit, wherein the temperature controlling unit outputs the temperature control signal according to the temperature data and a predetermined temperature.

7. The measurement apparatus for gas sensor according to claim 1, further comprising:
a probe module facing the wafer-holding module, wherein the probe module comprises:
a circuit board having a control circuit, a hole, and a heat conduction ring enclosing the hole, wherein the heat conduction ring is respectively connected to the circuit board and a heat dissipation module spatially isolated from the circuit board;
a gas inlet channel connecting the hole, and configured to allow a testing gas to be directed toward the holding carrier; and
a pin structure exposed out of the hole and comprising a plurality of probes, wherein each of the probes comprises a tip portion adapted to detect a surface electrode of the at least one uncut gas-sensing cell, and a cantilever portion in contact with the heat conduction ring.

8. The measurement apparatus for gas sensor according to claim 7, wherein the probes are made of a tungsten material.

9. The measurement apparatus for gas sensor according to claim 7, wherein each of the probes further comprises a protection layer made of a fluoro material.

10. The measurement apparatus for gas sensor according to claim 7, wherein the heat conduction ring is made of an epoxy resin material.
